(19) United States
(12) Patent Application Publication
(10) Pub. No.: US 2025/0221613 A1
(43) Pub. Date: Jul. 10, 2025

(54) CAMERA UNIT, IMAGE PICKUP MODULE, AND ENDOSCOPE

(71) Applicant: OLYMPUS MEDICAL SYSTEMS CORP., Tokyo (JP)

(72) Inventors: Takatoshi IGARASHI, Kamiina-gun (JP); Takahiro SHIMOHATA, Shiojiri-shi (JP)

(73) Assignee: OLYMPUS MEDICAL SYSTEMS CORP., Tokyo (JP)

(21) Appl. No.: 19/013,395

(22) Filed: Jan. 8, 2025

Related U.S. Application Data
(60) Provisional application No. 63/619,485, filed on Jan. 10, 2024.

(51) Int. Cl. A61B 1/05; A61B 1/00

(52) U.S. Cl. CPC A61B 1/051; A61B 1/00096; A61B 1/0011

(57) ABSTRACT

A camera unit includes an optical element comprising one or more lenses, an image sensor, and a second adhesive, a first resin, and a second resin. The image sensor includes a cover glass, a solid-state image pickup device, and a first adhesive that adheres the cover glass and the solid-state image pickup device. The first resin has a light-shielding property, and covers at least a part of a side surface of the optical element. The second resin covers a side surface of the first adhesive. A tensile elastic modulus of the second resin is smaller than a tensile elastic modulus of the first resin.

### RELATED APPLICATION DATA

This application is based on and claims priority under 35 U.S.C. § 119 to U.S. Provisional Application No. 63/619,485, filed on Jan. 10, 2024, the entire contents of which are incorporated herein by reference.

### BACKGROUND

### 1. Technical Field

The present disclosure relates to a camera unit configured by bonding an optical element and an image sensor, an image pickup module including the camera unit, and an endoscope including the image pickup module.

### 2. Description of the Related Art

Conventionally, an image pickup module including a camera unit has been used for an endoscope. The camera unit includes an optical element and an image sensor, for example. The image sensor is configured by bonding a cover glass and a solid-state image pickup device by an adhesive, for example.

For example, Japanese Patent Application Laid-Open Publication No. 2012-189788 discloses a camera module configured such that a camera module main body including a solid-state image pickup device and a WLO (wafer level optics) is covered with a light-shielding resin and a first shield configured to cut off electromagnetic waves. The first shield is made of metal formed in a bottomed cylinder shape. The camera module main body is housed in the first shield, with a gap. The gap between the camera module main body and the first shield is filled with the light-shielding resin. Furthermore, the publication discloses that the light-shielding resin is composed of an upper layer resin (thermosetting resin) and a lower layer resin (ultraviolet curing resin).

### SUMMARY

A camera unit according to one aspect of the present disclosure includes: an optical element comprising one or more lenses; an image sensor comprising a cover glass, a solid-state image pickup device, and a first adhesive that adheres the cover glass and the solid-state image pickup device; a first resin having a light-shielding property, the first resin covering at least a part of a side surface of the optical element; and a second resin that covers a side surface of the first adhesive. A tensile elastic modulus of the second resin is smaller than a tensile elastic modulus of the first resin.

An image pickup module according to one aspect of the present disclosure includes a camera unit, a three-dimensional wiring board, and a third resin. The camera unit includes: an optical element comprising one or more lenses; an image sensor including a cover glass, a solid-state image pickup device, and a first adhesive that adheres the cover glass and the solid-state image pickup device; a first resin having a light-shielding property, the first resin covering at least a part of a side surface of the optical element; and a second resin that covers a side surface of the first adhesive, and a tensile elastic modulus of the second resin is smaller than a tensile elastic modulus of the first resin. The three-dimensional wiring board includes a side wall and a bottom defining a cavity, the camera unit being disposed in the cavity and electrically connected to the bottom. The third resin has a light-shielding property and is located between at least the side surface of the camera unit and the side wall.

An endoscope according to one aspect of the present disclosure includes an insertion section, and an image pickup module. The image pickup module disposed at a distal end of the insertion section. The image pickup module includes: a camera unit, a three-dimensional wiring board, and a third resin. The camera unit includes: an optical element comprising one or more lenses; an image sensor including a cover glass, a solid-state image pickup device, and a first adhesive that adheres the cover glass and the solid-state image pickup device; a first resin having a light-shielding property, the first resin covering at least a part of a side surface of the optical element; and a second resin that covers a side surface of the first adhesive, and a tensile elastic modulus of the second resin is smaller than a tensile elastic modulus of the first resin. The three-dimensional wiring board includes a side wall and a bottom defining a cavity the camera unit being disposed in the cavity and electrically connected to the bottom. The third resin has a light-shielding property and is located between the side surface of the camera unit and the side wall.

### BRIEF DESCRIPTION OF THE DRAWINGS

FIG. 1 is a perspective view showing an appearance of an endoscope according to a first embodiment of the present disclosure.

FIG. 2 is a perspective view showing an outline of an image pickup module according to the first embodiment.

FIG. 3 is a view showing one example of a cross section, which is taken along the line III-III in FIG. 2, of the image pickup module according to the first embodiment.

FIG. 4 is a view showing another example of the cross section, which is taken along the line III-III in FIG. 2, of the image pickup module according to the first embodiment.

FIG. 5 is a table showing one example of physical properties of a first resin, a second resin, a third resin, and a first adhesive in the first embodiment.

FIG. 6 is a flowchart showing a manufacturing process of a camera unit according to the first embodiment.

FIG. 7 is a chart for describing the manufacturing process of the camera unit according to the first embodiment.

### DETAILED DESCRIPTION

In general, when a camera unit is connected to a wiring board of an image pickup module in a manufacturing process, soldering is performed by the solder bump being reflowed under a high temperature in a reflow furnace, for example. In addition, an endoscope to which an image pickup module is mounted is used under a high temperature, a low temperature, or high humidity in some cases. If a change occurs in at least one of the temperature and the humidity under which the camera unit is used, deformation occurs in a resin covering a side surface of the camera unit, and an image sensor may receive a stress from the resin in some cases. In such cases, a crack occurs in a layer of an adhesive between a cover glass and a solid-state image pickup device, from an outer circumference to an inner side of the image sensor, and the crack may deteriorate an image quality of an image.

According to the embodiment to be described below, it is possible to provide a camera unit, an image pickup module, and an endoscope, which are capable of preventing a crack from occurring in an adhesive by a stress of a resin which is generated due to a change in at least one of a temperature and humidity.

Hereinafter, an embodiment of the present disclosure will be described with reference to drawings. However, the present disclosure is not limited by the embodiment to be described below.

Note that the same or corresponding elements are attached with the same reference signs as appropriate in the drawings. Furthermore, care should be taken on the fact that the drawings are schematic, and a relationship between lengths of respective elements, a ratio of the length of one element to that of another element, a number of respective elements in one drawing may be different from the actual ones in some cases, for simplification of the description. The respective plurality of drawings include parts in which the relationships and ratios of the lengths, the number of the elements, and the like are different in some cases.

### First Embodiment

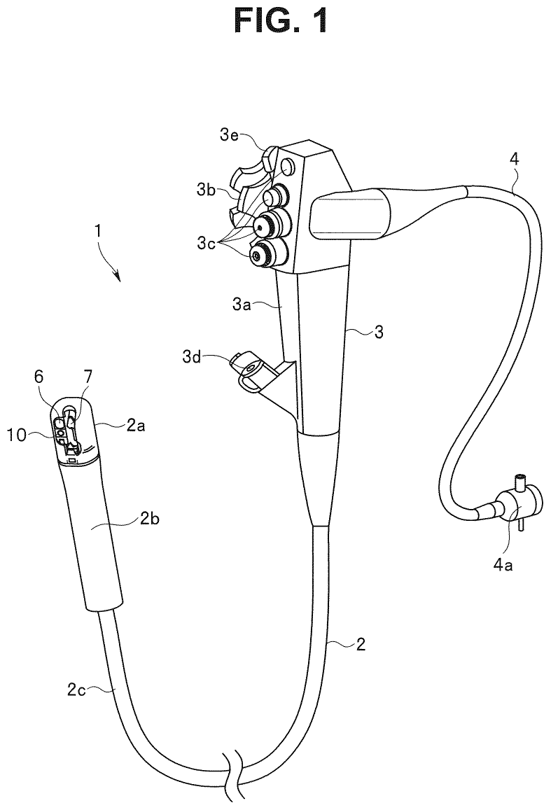

FIG. 1 to FIG. 7 show the first embodiment of the present disclosure. FIG. 1 is a perspective view showing an appearance of an endoscope 1 according to the first embodiment.

The endoscope 1 includes an insertion portion 2, an operation portion 3, and a universal cable 4. The endoscope 1 is configured as a lateral-viewing electronic endoscope, for example. Note that the endoscope 1 may be a front-viewing endoscope.

The insertion portion 2 is a part configured to be inserted into a subject. The subject may be a living body such as human being or animals, or a non-living body such as a machine, architecture, or the like. The insertion portion 2 includes, in the following order from the distal end side toward the proximal end side, a distal end portion 2a, a bending portion 2b, and a flexible tube portion 2c.

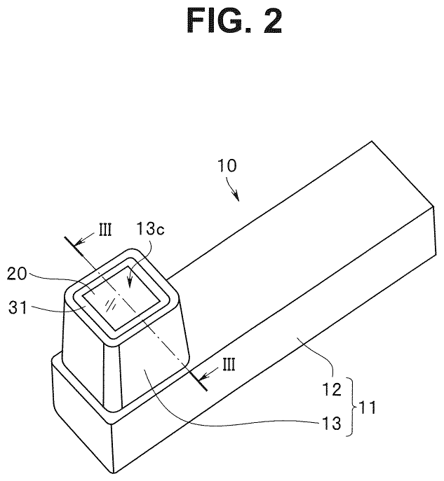

The distal end portion 2a includes an image pickup module 10 (see FIG. 2, etc.), an illumination unit 6, a raising base 7, and the like. Note that description is made here on the example in which the endoscope 1 includes the raising base 7, but the endoscope 1 may not include the raising base 7.

The bending portion 2b is a part configured to be bendable in two directions, or in four directions, i.e., up, down, left, and right directions, for example.

The flexible tube portion 2c is a tubular portion having flexibility. Note that a description is made here on an example in which the endoscope 1 is a flexible endoscope having the flexible tube portion 2c. However, the endoscope 1 may be a rigid endoscope in which a part corresponding to the flexible tube portion 2c is rigid.

The operation portion 3 is disposed on the proximal end side of the insertion portion 2. The operation portion 3 is a part for a user to operate the endoscope 1. The operation portion 3 includes a grasping portion 3a, a bending operation knob 3b, a plurality of operation buttons 3c, a treatment instrument insertion port 3d, and a treatment instrument raising lever 3e.

The grasping portion 3a is a part for a user to grasp the endoscope 1 with the palm.

The treatment instrument insertion port 3d is an opening on the proximal end side of the treatment instrument channel. A treatment instrument such as a forceps is inserted from the treatment instrument insertion port 3d into the treatment instrument channel. The distal end portion of the treatment instrument is guided from the treatment instrument channel to the raising base 7, to be protruded into a subject. Various kinds of treatment are performed on the subject with the protruded distal end portion of the treatment instrument.

The bending operation knob 3b is an operation device for operating the bending of the bending portion 2b. The bending operation knob 3b is operated with the thumb of the hand grasping the grasping portion 3a, for example. When the bending operation knob 3b is operated, bending operation wires, not shown, are pulled, and the bending portion 2b is bent.

When the bending portion 2b is bent, the direction of the distal end portion 2a is changed. Then, the direction of the image pickup to be performed by the image pickup module 10, and the irradiation direction of the illumination light from the illumination unit 6 are changed. In addition, the bending portion 2b is bent also for improving the insertion performance of the insertion portion 2 in the subject.

The plurality of operation buttons 3c include, for example, a gas/liquid feeding button, a suction button, and buttons related to image pickup.

The gas/liquid feeding button is a button for operating gas/liquid feeding to an observation window provided on the distal end surface of the image pickup module 10 in the distal end portion 2a. The observation window is cleaned by liquid feeding, and the liquid after the cleaning is blown away by gas feeding. The gas and the liquid are fed via a gas/liquid feeding channel.

The suction button is a button for performing operation of sucking inside the subject from the distal end portion 2a. The suction inside the subject is performed through the treatment instrument channel which serves also as a suction channel, for example. When the suction operation is performed, a liquid and a mucosa, for example, are sucked from the subject.

The buttons related to image pickup include button switches, for example, a freeze button for temporarily stopping a monitor screen, a release button for picking up a still image.

The treatment instrument raising lever 3e is a lever for operating raising and lowering of the raising base 7 in the distal end portion 2a.

The universal cable 4 is extended, for example, from a side surface on the proximal end side of the operation portion 3. A connector 4a is provided at an extension end of the universal cable 4. The connector 4a is configured to connect the endoscope 1 to an endoscope processor (video processor), a light source apparatus, a suction pump, a liquid feeding tank, and the like.

FIG. 2 is a perspective view showing an outline of the image pickup module 10 in the first embodiment.

The image pickup module 10 includes a three-dimensional wiring board 11 and a camera unit 20.

The three-dimensional wiring board 11 is configured as a three-dimensional (solid) MID (molded interconnect device). The three-dimensional wiring board 11 is configured such that a plurality of conductor patterns, electrodes, and the like are formed three-dimensionally in a curved surface, an uneven surface, and through holes of a three-dimensional substrate that is injection-molded, for example.

The three-dimensional wiring board 11 includes an assembly member 12 and a protrusion member 13 protruding from the assembly member 12.

The protrusion member 13 is a square cylinder having a bottom surrounded by a rectangular side wall, for example. An inside of the protrusion member 13 is formed as a cavity 13c in which a camera unit 20 is housed.

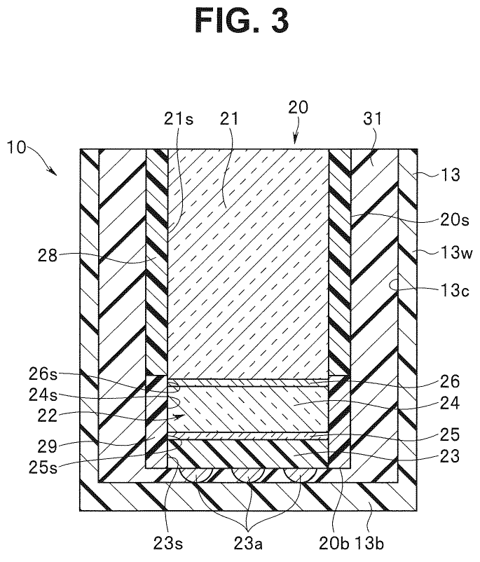

FIG. 3 is a view showing one example of a cross section, which is taken along the line III-III in FIG. 2, of the image pickup module 10 according to the first embodiment.

The camera unit 20 includes an optical element 21, an image sensor 22, a second adhesive 26, a first resin 28, and a second resin 29.

The optical element 21 is a stacked lens including a plurality of optical lenses, for example, although illustration of the specific configuration thereof is omitted. The optical element 21 is configured as WLO (wafer level optics) manufactured on a wafer by using a semiconductor manufacturing process, for example. The optical element 21 forms an optical image of incident light. The optical element may includes one or more lenses.

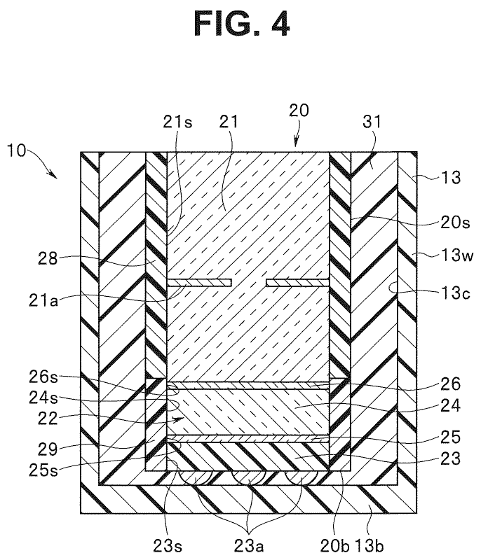

FIG. 4 is a view showing another example of the cross section, which is taken along the line III-III in FIG. 2, of the image pickup module 10 according to the first embodiment. As shown in FIG. 4, the optical element 21 includes an optical diaphragm 21a for cutting unnecessary light, for example. The optical diaphragm 21a may be configured as a light-shielding film (a specific example includes a chromium film) having an opening at the center thereof.

The image sensor 22 includes a cover glass 24, a solid-state image pickup device 23, and a first adhesive 25.

The solid-state image pickup device 23 is configured such that a plurality of pixels that photoelectrically convert incident light to generate electric signals are arranged on an image pickup surface. The solid-state image pickup device 23 converts the optical image of an object, which has been formed by the optical element 21, into an image pickup signal composed of the electric signal of each of the pixels. Examples of the solid-state image pickup device 23 include a CCD (charge-coupled device), a CMOS (complementary metal oxide semiconductor), and the like.

The solid-state image pickup device 23 includes a plurality of solder bumps 23a arranged on a surface opposite the image pickup surface, to configure a ball grid array.

The first adhesive 25 is a transparent optical adhesive. The first adhesive 25 adheres the cover glass 24 and the image pickup surface of the solid-state image pickup device 23. The cover glass 24 and the solid-state image pickup device 23 are adhered to each other by the first adhesive 25, to configure the image sensor 22.

The second adhesive 26 is a transparent optical adhesive. The second adhesive 26 adheres the optical element 21 and the cover glass 24. Thus, the optical element 21 is adhered to the image sensor 22.

The first resin 28 has a light-shielding property, and covers at least a part of a side surface 21s of the optical element 21. Note that, as a method for providing the light-shielding property to the resin, a method for adding a black pigment such as carbon black to the resin may be used, for example. However, the first resin 28 is not limited to black, as long as the first resin has the light-shielding property.

In the examples shown in FIG. 3 and FIG. 4, the first resin 28 covers up to a part slightly upper side (incident light side) than the second adhesive 26 in a whole circumference of the side surface 21s of the optical element 21.

In the example shown in FIG. 4 illustrating the optical diaphragm 21a, the first resin 28 covers from the incident light side to the part between the optical diaphragm 21a and the second adhesive 26 in the whole circumference of the side surface 21s of the optical element 21.

The second resin 29 covers a side surface 25s of the first adhesive 25. The second resin 29 further covers at least a part of a side surface 26s of the second adhesive 26.

In the examples shown in FIG. 3 and FIG. 4, the second resin 29 covers a whole circumference of a side surface 23s of the solid-state image pickup device 23, a whole circumference of the side surface 25s of the first adhesive 25, a whole circumference of a side surface 24s of the cover glass 24, a whole circumference of the side surface 26s of the second adhesive 26, and a whole circumference of a lower end side (solid-state image pickup device 23 side) of the side surface 21s of the optical element 21.

On the side surface 21s of the optical element 21, the first resin 28 and the second resin 29 are connected, and the whole circumference of the side surface 21s are covered with the first resin 28 and the second resin 29, with no gap.

The camera unit 20 is disposed in the cavity 13c of the protrusion member 13 of the three-dimensional wiring board 11.

The cavity 13c is formed by a side wall 13w and a bottom floor 13b, and is a space surrounded by an inner surface of the side wall 13w and an inner surface of the bottom floor 13b. The side wall 13w surrounds the camera unit 20, with a distance between itself and the side surface 20s of the camera unit 20.

The camera unit 20 is connected to an electrode, not shown, provided on the inner surface of the bottom floor 13b of the cavity 13c of the three-dimensional wiring board 11, with the solder bumps 23a. In other words, the inner surface of the bottom floor 13b is a bottom surface of the cavity 13c to which the camera unit 20 is connected. The camera unit 20 may be electrically connected to the electrode.

The solder bumps 23a are connected by reflow soldering, for example. The reflow soldering, as is known, is a soldering processing in which a solder paste, which is a mixture of grains of solder and flux, is heated in a reflow furnace to vaporize the flux with heat and join the grains of solder to each other.

The image pickup module 10 includes a third resin 31 that fills a space between the camera unit 20 and the cavity 13c. The third resin 31 has a light-shielding property. The third resin 31 is located between the side surface 20s of the camera unit 20 and the inner surface of the side wall 13w, and between the bottom surface 20b of the camera unit 20 and the inner surface of the bottom floor 13b.

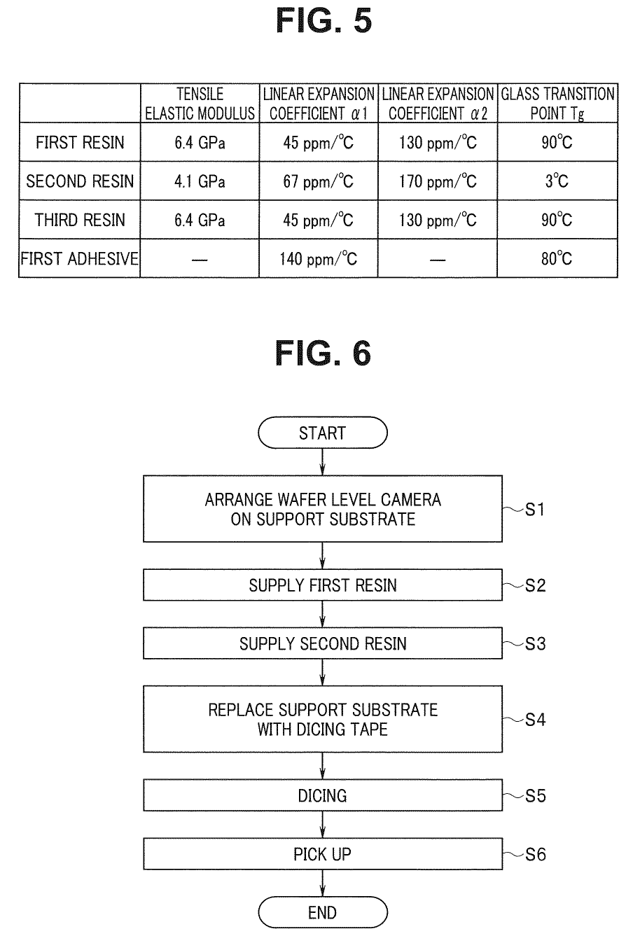

FIG. 5 is a table showing one example of physical properties of the first resin 28, the second resin 29, the third resin 31, and the first adhesive 25 in the first embodiment. Note that the sign “-” in the table in FIG. 5 indicates that no value is obtained.

In the example shown in FIG. 5, the tensile elastic modulus of the first resin 28 is 6.4 GPa, the tensile elastic modulus of the second resin 29 is 4.1 GPa. Thus, the tensile elastic modulus of the second resin 29 is smaller than the tensile elastic modulus of the first resin 28.

In addition, the tensile elastic modulus of the third resin 31 is 6.4 GPa, and is equal to the tensile elastic modulus of the first resin 28 in the example shown in FIG. 5. The third resin 31 is made of the same material as that of the first resin 28, for example. However, the third resin 31 may be made of a material (different resin) different from that of the first resin 28, as long as the material has a light-shielding property.

If the third resin 31 is made of a material having a tensile elastic modulus equal to or greater than the tensile elastic modulus of the first resin 28, the rigidity of the whole image pickup module 10 can be increased.

In addition, if the third resin 31 is made of a material having a tensile elastic modulus equal to or smaller than the tensile elastic modulus of the second resin 29, a stress to be applied from the third resin 31 to the cover glass 24 can be relaxed when a change occurs in at least one of a temperature and humidity.

Incidentally, in general, resins are brought into a hard glass state at temperatures below a glass transition point Tg (or equal to or below the glass transition point Tg) due to restricted molecular motion, and are brought into a soft rubber state at temperatures equal to or above the glass transition point Tg (or above the glass transition point Tg) due to easy molecular motion.

The coefficient of thermal expansion of each of the resins at the temperatures below the glass transition point Tg (or equal to or below the glass transition point Tg) is referred to as α1, and the coefficient of thermal expansion of each of the resins at the temperatures equal to or above the glass transition point Tg (or above the glass transition point Tg) is referred to as α2.

The coefficient of thermal expansion α1 is 45 ppm/° C. for the first resin 28, 67 ppm/° C. for the second resin 29, 45 ppm/° C. for the third resin 31, and 140 ppm/° C. for the first adhesive 25.

The coefficient of thermal expansion α2 is 130 ppm/° C. for the first resin 28, 170 ppm/° C. for the second resin 29, 130 ppm/° C. for the third resin 31.

The glass transition point Tg is 90° C. for the first resin 28, 3° C. for the second resin 29, 90° C. for the third resin 31, and 80° C. for the first adhesive 25.

First, the case where the temperature is below (or equal to or below) 3° C. is assumed. In the case of this temperature range, the coefficient of thermal expansions of the first resin 28, the second resin 29, the third resin 31, and the first adhesive 25 are all α1.

In this case, the difference (absolute value of the difference, the same applies hereafter) between the coefficient of thermal expansion α1 of the second resin 29, which is equal to 67 ppm/° C., and the coefficient of thermal expansion α1 of the first adhesive 25, which is equal to 140 ppm/° C., is 73 ppm/° C.

In addition, the difference between the coefficient of thermal expansion α1 of the first resin 28, which is equal to 45 ppm/° C., and the coefficient of thermal expansion α1 of the first adhesive 25, which is equal to 140 ppm/° C., is 95 ppm/° C.

Thus, in the case of the temperature below (or equal to or below) 3° C., the difference of 73 ppm/° C. between the coefficient of thermal expansion α1 of the second resin 29 and the coefficient of thermal expansion α1 of the first adhesive 25 is smaller than the difference of 95 ppm/° C. between the coefficient of thermal expansion α1 of the first resin 28 and the coefficient of thermal expansion α1 of the first adhesive 25.

Next, the case where the temperature is equal to or above (or above) 3° C. and below (or equal to or below) 80° C. is assumed. In the case of this temperature range, the coefficient of thermal expansions of the first resin 28, the third resin 31, and the first adhesive 25 are α1, and the coefficient of thermal expansion of the second resin 29 is α2.

In this case, the difference between the coefficient of thermal expansion α2 of the second resin 29, which is equal to 170 ppm/° C., and the coefficient of thermal expansion α1 of the first adhesive 25, which is equal to 140 ppm/° C., is 30 ppm/° C.

Furthermore, the difference between the coefficient of thermal expansion α1 of the first resin 28, which is equal to 45 ppm/° C., and the coefficient of thermal expansion α1 of the first adhesive 25, which is equal to 140 ppm/° C., is 95 ppm/° C.

Thus, in the case where the temperature is equal to or above (or above) 3° C. and below (or equal to or below) 80° C., the difference of 30 ppm/° C. between the coefficient of thermal expansion α2 of the second resin 29 and the coefficient of thermal expansion α1 of the first adhesive 25 is smaller than the difference of 95 ppm/° C. between the coefficient of thermal expansion α1 of the first resin 28 and the coefficient of thermal expansion α1 of the first adhesive 25.

Thus, in the case where the temperature is below (or equal to or below) 80° C., the following relationship is established, that is, the difference between the coefficient of thermal expansion of the second resin 29 and the coefficient of thermal expansion of the first adhesive 25 is smaller than the difference between the coefficient of thermal expansion of the first resin 28 and the coefficient of thermal expansion of the first adhesive 25.

As examples of materials that satisfy the physical properties shown in FIG. 5, the first resin 28 and the third resin 31 may be an epoxy resin, and the second resin 29 may be an acrylic resin, and the first adhesive 25 may be an epoxy resin.

However, the materials are not limited to the examples, but an acrylic resin or a silicone resin may be used for the first resin 28 and the third resin 31. The epoxy resin or the silicone resin may be used for the second resin 29. The acrylic resin or the silicone resin may be used for the first adhesive 25.

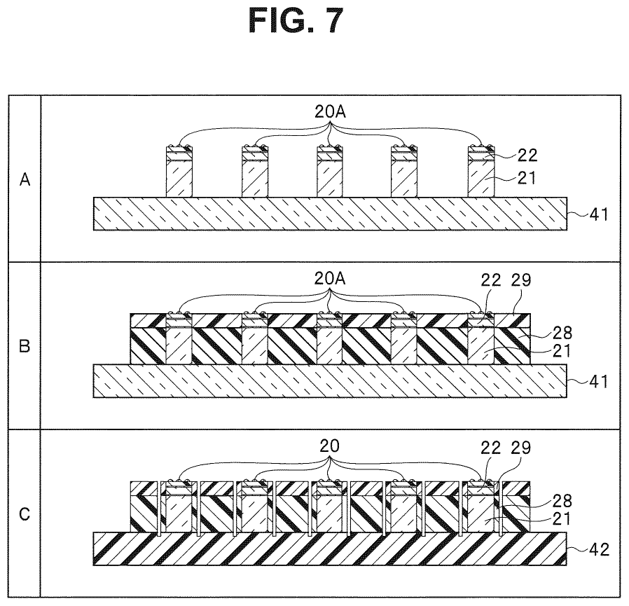

FIG. 6 is a flowchart showing a manufacturing process of the camera unit 20 according to the first embodiment. FIG. 7 is a chart for describing the manufacturing process of the camera unit 20 according to the first embodiment.

The naked camera unit 20, which has been manufactured by a semiconductor manufacturing process and to which the first resin 28 and the second resin 29 have not been applied yet, will be referred to as a wafer level camera 20A. The wafer level camera 20A includes the optical element 21, the image sensor 22, and the second adhesive 26.

FIG. 6 and FIG. 7 show the process of manufacturing the camera unit 20 by using the already manufactured wafer level cameras 20A.

When the processing shown in FIG. 6 starts, the wafer level cameras 20A are arranged on a support substrate 41 so as to be spaced apart from each other, as shown in the A column of FIG. 7 (step S1). The support substrate 41 is configured by a glass substrate, for example. Each of the wafer level cameras 20A is arranged such that the incident light side thereof is temporarily adhered to the support substrate 41 (up and down positions are reversed from the state shown in FIG. 3).

The first resin 28 is supplied to each space between the wafer level cameras 20A arranged on the support substrate 41 (and outsides of the respective wafer level camera 20A arranged on both ends, the same applies hereafter), so as to reach up to the height described with reference to FIG. 3 (in FIG. 7, the height slightly lower (incident light side) than the second adhesive 26 (step S2).

Furthermore, the second resin 29 is supplied onto the first resin 28, so as to reach up to the height described with reference to FIG. 3 (height covering the whole circumference of the side surface 23s of the solid-state image pickup device 23 of the image sensor 22) (step S3).

After the processing in the step S3 ends, as shown in the B column in FIG. 7, the whole circumference of the side surface of each of the wafer level cameras 20A is covered with the first resin 28 and the second resin 29. In the state shown in the B column in FIG. 7, a plurality of wafer level cameras 20A are integrated by the first resin 28 and the second resin 29.

In this state, the support substrate 41 is detached from the plurality of integrated wafer level cameras 20A, to be replaced with a dicing tape 42 (step S4).

Cutting is performed such that the first resin 28 and the second resin 29 remain in a predetermined thickness on the side surface of each of the wafer level cameras 20A (dicing) (step S5). Then, as shown in the C column in FIG. 7, a plurality of camera units 20 obtained by dicing are adhered on the dicing tape 42.

After that, each of the camera units 20 is detached from the dicing tape 42 to be picked up (step S6), to terminate the processing shown in FIG. 6.

The picked-up camera unit 20 is connected to the three-dimensional wiring board 11 by reflow soldering, as already described above.

According to the first embodiment, the tensile elastic modulus of the second resin 29 covering the side surface 25s of the first adhesive 25 is made to be smaller than the tensile elastic modulus of the first resin 28. With such a configuration, the stress to be applied from the second resin 29 to the first adhesive 25 due to the change in at least one of the temperature and humidity can be decreased, compared with the case where the side surface 25s is covered with the first resin 28. This can prevent a crack in the first adhesive 25 from occurring by the stress of the resin covering the side surface of the wafer level camera 20A, the stress being generated due to the change in at least one of the temperature and humidity.

Specifically, during the manufacturing in which the camera unit 20 is connected to the three-dimensional wiring board 11 by the reflow soldering under a high temperature, and during the use of the endoscope 1 under the environment of a high temperature, a low temperature, or high humidity, the stress to be applied to the first adhesive 25 can be reduced, to thereby prevent the crack from occurring in the first adhesive 25.

Furthermore, the difference between the coefficient of thermal expansion of the second resin 29 and the coefficient of thermal expansion of the first adhesive 25 is made to be smaller than the difference between the coefficient of thermal expansion of the first resin 28 and the coefficient of thermal expansion of the first adhesive 25. With such a configuration, the difference between a change rate in the length of the second resin 29 and a change rate in the length of the first adhesive 25 due to a temperature change becomes smaller than the difference between a change rate in the length of the first resin 28 and the change rate in the length of the first adhesive 25 due to the temperature change. Therefore, by covering the side surface 25s of the first adhesive 25 with the second resin 29, the occurrence of a crack in the first adhesive 25 can be prevented.

Furthermore, the second resin 29 further covers at least a part of the side surface 26s of the second adhesive 26. Therefore, during the manufacturing and the use, the stress to be applied to the second adhesive 26 can be reduced, to thereby be capable of preventing a crack from occurring in the second adhesive 26.

The side surface on the incident light side of the optical element 21 is covered with the first resin 28 having the light-shielding property, to thereby be capable of preventing unnecessary light from entering the optical element 21.

According to the manufacturing method of the camera unit 20 described with reference to FIG. 6 and FIG. 7, a plurality of camera units 20 can be manufactured collectively. Such a method eliminates a need for performing the processing of sealing each of the singulated camera units 20 with the resins one by one. The method enables the plurality of camera units 20 to be manufactured by the automated and mechanized processes, to thereby be capable of reducing the manufacturing costs.

Note that the present disclosure is not limited to the above-described embodiment as it is. The present disclosure can be embodied by modifying the constituent elements in a range without departing from the gist of the disclosure at the practical stage. In addition, various aspects of the disclosure can be achieved by appropriately combining the plurality of constituent elements disclosed in each of the above-described embodiment. Some of the constituent elements may be deleted from all the constituent elements shown in the embodiment, for example. Furthermore, constituent elements of different embodiments may be combined as appropriate. It goes without saying that various modifications and applications can be implemented within a range without departing from the gist of the disclosure.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

### EXAMPLE

1. A camera unit comprising:
   an optical element;
   an image sensor comprising a cover glass, a solid-state image pickup device, and a first adhesive that adheres the cover glass and the solid-state image pickup device;
   a second adhesive that adheres the optical element and the cover glass;
   a first resin having a light-shielding property, the first resin covering at least a part of a side surface of the optical element; and
   a second resin that covers a side surface of the first adhesive, wherein
   a tensile elastic modulus of the second resin is smaller than a tensile elastic modulus of the first resin.

What is claimed is:

1. A camera unit comprising:
an optical element comprising one or more lenses;
an image sensor comprising a cover glass, a solid-state image pickup device, and a first adhesive adhering the cover glass and the solid-state image pickup device;
a first resin having a light-shielding property, the first resin covering at least a part of a side surface of the optical element; and
a second resin covering a side surface of the first adhesive; wherein
a tensile elastic modulus of the second resin is smaller than a tensile elastic modulus of the first resin.

2. The camera unit according to claim 1, wherein
the second resin further covers a side surface of the cover glass and a side surface of the solid-state image pickup device.

3. The camera unit according to claim 2, further comprising:
a second adhesive adhering the optical element and the cover glass; and wherein the second resin further covers at least a part of a side surface of the second adhesive.

4. The camera unit according to claim 1, wherein
a difference between a coefficient of thermal expansion of the second resin and a coefficient of thermal expansion of the first adhesive is smaller than a difference between a coefficient of thermal expansion of the first resin and the coefficient of thermal expansion of the first adhesive.

5. The camera unit according to claim 1, wherein
the optical element includes an optical diaphragm, and
the first resin covers the side surface of the optical element, from an incident light side to a position between the optical diaphragm and the second adhesive in an optical axis direction.

6. An image pickup module comprising,
a camera unit comprising:
an optical element comprising one or more lenses;
an image sensor comprising a cover glass, a solid-state image pickup device, and a first adhesive adhering the cover glass and the solid-state image pickup device;
a first resin having a light-shielding property, the first resin covering at least a part of a side surface of the optical element; and
a second resin covering a side surface of the first adhesive; wherein
a tensile elastic modulus of the second resin is smaller than a tensile elastic modulus of the first resin;
a three-dimensional wiring board including a side wall and a bottom defining a cavity, the camera unit being disposed in the cavity and electrically connected to the bottom; and
a third resin having a light-shielding property and being located between at least the side surface of the camera unit and the side wall.

7. The image pickup module according to claim 6, wherein
the second resin further covers a side surface of the cover glass and a side surface of the solid-state image pickup device.

8. The image pickup module according to claim 7, further comprising:
a second adhesive adhering the optical element and the cover glass; and wherein the second resin further covers at least a part of a side surface of the second adhesive.

9. The image pickup module according to claim 6, wherein
a difference between a coefficient of thermal expansion of the second resin and a coefficient of thermal expansion of the first adhesive is smaller than a difference between a coefficient of thermal expansion of the first resin and the coefficient of thermal expansion of the first adhesive.

10. The image pickup module according to claim 6, wherein
the optical element includes an optical diaphragm, and
the first resin covers the side surface of the optical element, from an incident light side to a position between the optical diaphragm and the second adhesive.

11. The image pickup module according to claim 6, wherein
a tensile elastic modulus of the third resin is equal to the tensile elastic modulus of the first resin.

12. The image pickup module according to claim 11, wherein
the third resin is made of a same material as that of the first resin.

13. The image pickup module according to claim 12, wherein
the first resin and the third resin are epoxy resins, and the second resin is an acrylic resin.

14. An endoscope comprising,
an insertion section; and
an image pickup module disposed at a distal end of the insertion section, the image pickup module comprising:
a camera unit comprising:
an optical element comprising one or more lenses;
an image sensor comprising a cover glass, a solid-state image pickup device, and a first adhesive adhering the cover glass and the solid-state image pickup device;
a first resin having a light-shielding property, the first resin covering at least a part of a side surface of the optical element; and
a second resin covering a side surface of the first adhesive; wherein
a tensile elastic modulus of the second resin is smaller than a tensile elastic modulus of the first resin;
a three-dimensional wiring board including a side wall and a bottom defining a cavity, the camera unit being disposed in the cavity and electrically connected to the bottom; and
a third resin having a light-shielding property and being located between the side surface of the camera unit and the side wall.

15. The endoscope according to claim 14, wherein
the second resin further covers a side surface of the cover glass and a side surface of the solid-state image pickup device.

16. The endoscope according to claim 15, further comprising
a second adhesive adhering the optical element and the cover glass; and wherein the second resin further covers at least a part of a side surface of the second adhesive.

17. The endoscope according to claim 14, wherein
a difference between a coefficient of thermal expansion of the second resin and a coefficient of thermal expansion of the first adhesive is smaller than a difference between a coefficient of thermal expansion of the first resin and the coefficient of thermal expansion of the first adhesive.

18. The endoscope according to claim 14, wherein
the optical element includes an optical diaphragm, and
the first resin covers the side surface of the optical element, from an incident light side to a position between the optical diaphragm and the second adhesive.

19. The endoscope according to claim 14, wherein
a tensile elastic modulus of the third resin is equal to the tensile elastic modulus of the first resin.

20. The endoscope according to claim 19, wherein
the third resin is made of a same material as that of the first resin.
